(19) United States
(12) Patent Application Publication
(10) Pub. No.: US 2017/0333509 A1
(43) Pub. Date: Nov. 23, 2017

(54) MAILLARD REACTION PRODUCT-DECOMPOSING AGENT

(71) Applicant: ARKRAY, Inc., Kyoto (JP)

(72) Inventors: Masako SHOSHIHARA, Kyoto-shi (JP); Hiroshige KAWAI, Kyoto-shi (JP)

(73) Assignee: ARKRAY, Inc., Kyoto (JP)

(21) Appl. No.: 15/601,709

(22) Filed: May 22, 2017

(30) Foreign Application Priority Data
May 23, 2016 (JP) 2016-102701
May 22, 2017 (JP) 2017-100807

(51) Int. Cl. A61K 36/53; A61K 8/73; A23L 5/10; A61K 36/235; A61K 36/00

(52) U.S. Cl. CPC A61K 36/53; A61K 36/235; A61K 36/00; A23L 5/19; A23L 5/10; A61K 8/732

(57) ABSTRACT

A Maillard reaction product-decomposing agent as well as a beverage composition, a food composition, a functional food, a cosmetic, a supplement, a quasi drug, or a pharmaceutical product that contains the agent as an active ingredient. A Maillard reaction product-decomposing agent containing an extract of fennel as an active ingredient. A beverage composition, a food composition, a functional food, a cosmetic, a supplement, a quasi drug, or a pharmaceutical product that contains, as an active ingredient, a Maillard reaction product-decomposing agent containing an extract of fennel as an active ingredient.

### CROSS REFERENCE TO RELATED APPLICATIONS

This application claims benefit of priority to Japanese Patent Application 2016-102701 filed May 23, 2016, and to Japanese Patent Application No. 2017-100807 filed May 22, 2017, the entire content of which is incorporated herein by reference.

### TECHNICAL FIELD

The present disclosure relates to a Maillard reaction product-decomposing agent as well as a beverage composition, a food composition, a functional food, a cosmetic, a supplement, a quasi drug, or a pharmaceutical product containing the same.

### BACKGROUND

It has been known that oxidation reactions and glycation reactions in a living body adversely affect cells and tissues. For example, proteins such as collagen are glycated by a glycation reaction (a Maillard reaction) in a living body and thereby the amino acids contained in the proteins become AGEs (advanced glycation end products) or AGEs form cross-linked structures between proteins. Thus, the accumulation of AGEs in proteins by the Maillard reaction and the formation of cross-linked structures by AGEs cause diseases such as diabetic complications, Alzheimer's disease, cataract, and arteriosclerosis as well as aging and functional decline of tissues such as the skin.

Known drugs for decomposing AGEs include N-phenacylthiazolium bromide (PTB) and N-phenacyl-4,5-dimethylthiazolium bromide (ALT-711) (Sara Vasan et al., Nature, 382, pp 275-278 (1996), and Sara Vasan et al., Archives of Biochemistry and Biophysics, 419, 89-96 (2003)). However, they are not used in terms of stability and side effects. In addition, for example, a lotus leaf extract and an Astilbe thunbergii extract have been studied (JP2007-119373A).

### SUMMARY

As described above, it has been considered that the accumulation of AGEs associated with the Maillard reaction and the cross-linked structures formed associated therewith may cause various diseases including the aging progress, diabetic complications, etc. Therefore, there are demands for a new substance that can decompose the products formed by the Maillard reaction, such as AGEs and intermediates thereof.

The present disclosure provides a new Maillard reaction product-decomposing agent as well as a beverage composition, a food composition, a functional food, a cosmetic, a supplement, a quasi drug, or a pharmaceutical product that contains the agent as an active ingredient.

As an aspect, the present disclosure relates to a Maillard reaction product-decomposing agent containing an extract of fennel as an active ingredient.

As another aspect, the present disclosure relates to a beverage composition, a food composition, a functional food, a cosmetic, a supplement, a quasi drug, or a pharmaceutical product that contains, as an active ingredient, a Maillard reaction product-decomposing agent of the present disclosure.

As another aspect, the present disclosure relates to a beverage composition, a food composition, a functional food, a cosmetic, a supplement, a quasi drug, or a pharmaceutical product for enhancing a Maillard reaction product-decomposing activity, each of which contains an extract of fennel as an active ingredient.

In an aspect, the present disclosure can provide a new Maillard reaction product-decomposing agent as well as a beverage composition, a food composition, a functional food, a cosmetic, a supplement, a quasi drug, or a pharmaceutical product that can enhance a Maillard reaction product-decomposing activity.

### DETAILED DESCRIPTION

The present disclosure is based on a new finding that water extracts of fennel (scientific name: Foeniculum vulgare), lemon balm (scientific name: Melissa officinalis), and rosemary (scientific name: Rosmarinus officinalis) each have an ability of breaking the cross-linked structures formed by the Maillard reaction, which is higher than that of PTB known as a Maillard reaction cross-link breaker, and particularly, a water extract of fennel, especially a hot water extract thereof, has the highest ability of breaking the cross-linked structures formed by the Maillard reaction.

Maillard Reaction Product-Decomposing Agent

As an aspect, the present disclosure relates to a Maillard reaction product-decomposing agent containing an extract of fennel as an active ingredient (a Maillard reaction product-decomposing agent of the present disclosure). In one or more embodiments, the Maillard reaction product-decomposing agent of the present disclosure can provide an effect of being capable of decomposing a product formed by the Maillard reaction (a Maillard reaction product), preferably an effect of being capable of decomposing a product formed by an amino-carbonyl reaction (also referred to as a Maillard reaction or a glycation reaction) between an amino group of amino acid, peptide, or protein and a sugar-derived carbonyl group in a living body.

In one or more embodiments, the Maillard reaction product-decomposing agent of the present disclosure can inhibit the accumulation of AGEs and can break the cross-linked structures of proteins, etc. that are formed by AGEs. Consequently, the diseases, aging of tissues, and functional decline of tissues that are caused by such accumulation and cross-linked structures can be prevented, improved, or treated. In one or more embodiments, the Maillard reaction product-decomposing agent of the present disclosure can improve metabolism.

Furthermore, the Maillard reaction product-decomposing agent of the present disclosure can provide an effect of being excellent in safety and productivity since an extract of fennel, which is a plant, is used therein as an active ingredient.

In the present disclosure, the term “Maillard reaction product” refers to a substance produced by the Maillard reaction. In one or more embodiments, substances produced by the Maillard reaction include an intermediate produced by the Maillard reaction and a final product produced by the Maillard reaction. In one or more embodiments, examples of the Maillard reaction product include polypeptides and proteins containing AGEs, polypeptides and proteins having cross-linked structures formed by AGEs, as well as glycated proteins produced by the Maillard reaction. In one or more embodiments, examples of the intermediate include substances in which the Maillard reaction is in progress and include, as non-limiting examples, glyoxal, methylglyoxal, and 3-deoxyglucosone. In one or more embodiments, examples of AGEs include amino acids and dipeptides glycated by the Maillard reaction, and non-limiting examples thereof include pentosidine, crossline, pyrropyridine, pyrraline, carboxymethyllysine (CML), carboxyethyllysine, carboxymethylarginine (CMA), argpyrimidine, an imidazolone compound, glyoxal-lysine dimer (GOLD), and methyl glyoxal-lysine dimer (MOLD).

In one or more embodiments, the Maillard reaction product-decomposing agent of the present disclosure provides an effect of allowing a Maillard reaction product to be decomposed. In the present disclosure, examples of “decomposing a Maillard reaction product” include, in one or more embodiments, decomposing intermediates formed associated with the Maillard reaction or breaking cross-linked structures formed between polypeptides and/or proteins by AGEs. In one or more embodiments, examples of “decomposing a Maillard reaction product” may further include decomposing polypeptides and proteins that contain AGEs, polypeptides and proteins having cross-linked structures formed by AGEs, or glycated proteins produced by the Maillard reaction. Furthermore, in one or more embodiments that are not particularly limited, examples of “decomposing a Maillard reaction product” may include breaking (cleaving) the C—C bonds in diketone structures contained in polypeptides and proteins that contain AGEs, polypeptides and proteins having cross-linked structures formed by AGEs, or glycated proteins produced by the Maillard reaction.

Furthermore, in one or more embodiments, the Maillard reaction product-decomposing agent of the present disclosure can decompose collagen cross-link (detrimental cross-link or aging cross-link) formed in a collagen molecule by, for example, glycation or oxidation. Thus, in one or more embodiments, the Maillard reaction product-decomposing agent of the present disclosure can inhibit the accumulation of AGEs in collagen.

In one or more embodiments, the Maillard reaction product-decomposing agent of the present disclosure may further contain, as an active ingredient, an extract of at least one of lemon balm and rosemary in terms of further improving the ability of breaking the cross-linked structure formed by the Maillard reaction. Moreover, preferably the Maillard reaction product-decomposing agent of the present disclosure contains at least one of lemon balm and fenugreek in terms of promoting the breakage of the collagen cross-link.

In one or more embodiments, the Maillard reaction product-decomposing agent of the present disclosure may further contain, as an active ingredient, an extract of at least one of fenugreek (scientific name: Trigonella foenum-graecum) and hibiscus (scientific name: Hibiscus sabdariffa).

When the Maillard reaction product-decomposing agent of the present disclosure contains an extract of another plant in addition to fennel, they may be blended at the same ratio (1 part by weight with respect to 1 part by weight of the extract of fennel) or they may be blended at different ratios.

In one or more embodiments, the Maillard reaction product-decomposing agent of the present disclosure contains an extract of fennel and an extract of lemon balm. In the present aspect, the blending ratio (the dry weight ratio) of the extract of fennel and the extract of lemon balm is, per 1 part by weight of the extract of fennel, 0.1 to 10 parts by weight of the extract of lemon balm, and 0.2 to 5 parts by weight of the extract of lemon balm in terms of further improving the ability of breaking the cross-linked structure formed by the Maillard reaction and promoting the breakage of the collagen cross-link.

In one or more embodiments, the Maillard reaction product-decomposing agent of the present disclosure contains an extract of fennel and an extract of rosemary. In the present aspect, the blending ratio (the dry weight ratio) of the extract of fennel and the extract of rosemary is, per 1 part by weight of the extract of fennel, 0.1 to 10 parts by weight of the extract of rosemary, and 0.2 to 5 parts by weight of the extract of rosemary in terms of further improving the ability of breaking the cross-linked structure formed by the Maillard reaction.

In one or more embodiments, the Maillard reaction product-decomposing agent of the present disclosure contains an extract of fennel and an extract of fenugreek. In the present aspect, the blending ratio (the dry weight ratio) of the extract of fennel and the extract of fenugreek is, per 1 part by weight of the extract of fennel, 0.1 to 10 parts by weight of the extract of fenugreek, and 0.2 to 5 parts by weight of the extract of fenugreek in terms of promoting the breakage of the collagen cross-link.

In one or more embodiments, the Maillard reaction product-decomposing agent of the present disclosure contains an extract of fennel, an extract of lemon balm, an extract of rosemary, and an extract of fenugreek. In the present aspect, the blending ratio (the dry weight ratio) of the extract of fennel, the extract of lemon balm, the extract of rosemary, and the extract of fenugreek is, per 1 part by weight of the extract of fennel, 0.05 to 20 parts by weight of the extract of lemon balm, 0.05 to 20 parts by weight of the extract of rosemary and 0.05 to 20 parts by weight of the extract of fenugreek, and 0.1 to 10 parts by weight of the extract of lemon balm, 0.1 to 10 parts by weight of the extract of rosemary and 0.1 to 10 parts by weight of the extract of fenugreek in terms of further improving the ability of breaking the cross-linked structure formed by the Maillard reaction and promoting the breakage of the collagen cross-link.

The extracts may be extracted from any parts of plants. In one or more embodiments, examples of the part to be used include whole plants, flowers, leaves, seeds, fruits, branches, stems, roots, calyxes, and bracts. The part to be used may be of one type, or two or more types of parts may be combined for use. In one or more embodiments, fennel is preferably extracted from seeds. In one or more embodiments, rosemary is preferably extracted from leaves. In one or more embodiments, lemon balm is preferably extracted from leaves. In one or more embodiments, fenugreek is preferably extracted from seeds. In one or more embodiments, hibiscus is preferably extracted from calyxes and bracts.

The extraction method is not particularly limited, but in one or more embodiments, examples thereof include solvent extraction. In one or more embodiments, examples of the extractant (solvent) include water, alcohols such as methanol and ethanol, and ether. In one or more embodiments, examples of the extract include a water extract and an organic solvent extract. In one or more embodiments, examples of the water extract include a hot water extract. In one embodiment, the solvent extracting is performed at a temperature of 60-100° C., 70-90° C., or 80° C., for a time period of 30 minutes to 5 hours, 2-6 hours, 3-5 hours, or 1 hour. In a preferred embodiment, hot water extraction is carried out at a temperature of 60-100° C., 70-90° C., or 80° C., for a time period of 30 minutes to 5 hours, 2-6 hours, 3-5 hours, or 1 hour. In one or more embodiments, examples of the form of the extract include an aqueous solution, a concentrated liquid, and a dried product. In one embodiment, drying is carried out by heat-treating the extract obtained by the solvent-extracting at a temperature of 70-150° C. such as 110° C. for 1-10 hours such as 4 hours.

A suitable method for producing a Maillard reaction product-decomposing agent according to the present invention is as follows: A defined amount of dry powder of fennel (such as 2 g) is extracted with a suitable amount of distilled water (such as 40 mL) at a temperature of 80° C. for one hour; followed by cooling to normal temperature. Thereafter, the obtained slurry is filtrated and the filtrate thus obtained is used as a plant extract. If a dried plant extract shall be used, the obtained plant extract is put into an aluminum tray and dried for e.g. four hours in an incubator set at e.g. 110° C.

The form of the Maillard reaction product-decomposing agent of the present disclosure is not particularly limited, but in one or more embodiments, examples thereof include solids, granules, powders, pastes, and liquids.

In one or more embodiments, the Maillard reaction product-decomposing agent of the present disclosure may contain other extracts and other components as long as they do not inhibit the AGEs decomposing functions of the active ingredients.

In one or more embodiments, the Maillard reaction product-decomposing agent of the present disclosure can be used as a raw material for a beverage composition, a food composition, a functional food, a cosmetic, a supplement, a feed, a quasi drug, a pharmaceutical product, etc. The form of the raw material is not particularly limited, but in one or more embodiments, examples thereof include solids, granules, powders, pastes, and liquids.

As one or more embodiments, the present disclosure relates to a beverage composition, a food composition, a functional food, a cosmetic, a supplement, a feed, a quasi drug, or a pharmaceutical product that contains a Maillard reaction product-decomposing agent of the present disclosure.

The intake per day of the Maillard reaction product-decomposing agent of the present disclosure is not particularly limited, but in one or more embodiments, it is at least 5 mg or not more than 2000 mg.

Method of Producing Maillard Reaction Product-Decomposing Agent

In another aspect, the present disclosure relates to a method of producing a Maillard reaction product-decomposing agent for decomposing a Maillard reaction product, the method including extracting fennel with a solvent. According to the production method of the present disclosure, the Maillard reaction product-decomposing agent of the present disclosure can be produced.

In one or more embodiments, the method of producing a Maillard reaction product-decomposing agent of the present disclosure may further include extracting at least one plant selected from the group consisting of lemon balm, rosemary, fenugreek, and hibiscus with a solvent.

In one or more embodiments, the production method of the present disclosure may include solvent-extracting a dried plant.

In one or more embodiments, the production method of the present disclosure may include filtrating or centrifuging an extract obtained by solvent-extracting.

In one or more embodiments, the production method of the present disclosure may include drying a resultant extract.

In one or more embodiments, the production method of the present disclosure may include mixing an extract of fennel and an extract of a plant other than fennel to obtain a mixture of the extracts. In one or more embodiments, the mixing ratios thereof may be the same (1 part by weight with respect to 1 part by weight of the extract of fennel) or may be different from each other.

In one or more embodiments, the production method of the present disclosure may include mixing an extract of fennel and an extract of lemon balm so that the extract of lemon balm is 0.1 to 10 parts by weight per 1 part by weight of the extract of fennel, and in terms of producing an Maillard reaction product-decomposing agent that can further improve the ability of breaking the cross-linked structure formed by the Maillard reaction and/or can promote the breakage of the collagen cross-link, the production method of the present disclosure may include mixing these extracts so that the extract of lemon balm is 0.2 to 5 parts by weight per 1 part by weight of the extract of fennel.

In one or more embodiments, the production method of the present disclosure may include mixing an extract of fennel and an extract of rosemary so that the extract of rosemary is 0.1 to 10 parts by weight per 1 part by weight of the extract of fennel, and in terms of producing an Maillard reaction product-decomposing agent that can further improve the ability of breaking the cross-linked structure formed by the Maillard reaction, the production method of the present disclosure may include mixing these extracts so that the extract of rosemary is 0.2 to 5 parts by weight per 1 part by weight of the extract of fennel.

In one or more embodiments, the production method of the present disclosure may include mixing an extract of fennel and an extract of fenugreek so that the extract of fenugreek is 0.1 to 10 parts by weight per 1 part by weight of the extract of fennel, and in terms of producing an Maillard reaction product-decomposing agent that can promote the breakage of the collagen cross-link, the production method of the present disclosure may include mixing these extracts so that the extract of fenugreek is 0.2 to 5 parts by weight per 1 part by weight of the extract of fennel.

In one or more embodiments, the production method of the present disclosure may include mixing an extract of fennel, an extract of lemon balm, and an extract of rosemary so that, per 1 part by weight of the extract of fennel, the extract of lemon balm is 0.05 to 20 parts by weight, the extract of rosemary is 0.05 to 20 parts by weight and the extract of fenugreek is 0.05 to 20 parts by weight, and in terms of producing an Maillard reaction product-decomposing agent that can further improve the ability of breaking the cross-linked structure formed by the Maillard reaction and/or can promote the breakage of the collagen cross-link, the production method of the present disclosure may include mixing these extracts so that, per 1 part by weight of the extract of fennel, the extract of lemon balm is 0.1 to 10 parts by weight, the extract of rosemary is 0.1 to 10 parts by weight and the extract of fenugreek is 0.1 to 10 parts by weight.

In one or more embodiments, the production method of the present disclosure may include mixing fennel and a plant other than fennel and solvent-extracting the resultant mixture to obtain a mixed extract. In one or more embodiments, the mixing ratios thereof may be the same (1 part by weight with respect to 1 part by weight of the fennel) or may be different from each other. Furthermore, they may be mixed together so that the ratios of the extracts of the respective plants in the resultant mixed extract are the same (1 part by weight with respect to 1 part by weight of the extract of fennel) or they may be mixed together so that the ratios thereof are different from each other.

Method of Producing Beverage Composition, Food Composition, Functional Food, Cosmetic, Supplement, Quasi Drug, or Pharmaceutical Product

In another aspect, the present disclosure relates to a method of producing a beverage composition, a food composition, a functional food, a cosmetic, a supplement, a quasi drug, or a pharmaceutical product (a method of producing a beverage composition, etc. of the present disclosure). The method of producing a beverage composition, etc. of the present disclosure includes adding an extract of fennel to a beverage composition, a food composition, a functional food, a cosmetic, a supplement, a quasi drug, or a pharmaceutical product to provide it with a Maillard reaction product-decomposing effect.

In one or more embodiments, the method of producing a beverage composition, etc. of the present disclosure may further include adding an extract of at least one plant selected from the group consisting of lemon balm, rosemary, fenugreek, and hibiscus to a beverage composition, a food composition, a functional food, a cosmetic, a supplement, a quasi drug, or a pharmaceutical product to provide it with a Maillard reaction product-decomposing effect.

In one or more embodiments, examples of the extract include a water extract and an organic solvent extract of each plant mentioned above. In one or more embodiments, examples of the water extract include a hot water extract. In one or more embodiments, examples of the form of the extract include an aqueous solution, a concentrated liquid, and a dried product.

Extract Composition

In another aspect, the present disclosure relates to a fennel extract composition for enhancing the Maillard reaction product-decomposing function. In the present disclosure, the term “extract composition” refers to a liquid or a solid that is obtained by an extraction treatment of a plant. In one or more embodiments, examples of the extract composition include a supernatant and a filtrate that are obtained by solvent-extracting a plant. In one or more embodiments, examples of the extractant include water, alcohols such as methanol and ethanol, and ether. In one or more embodiments, examples of the form of the extract composition include an aqueous solution, a concentrated liquid, and a dried product. In one or more embodiments, the extract composition of the present disclosure can be used as a food raw material. In the present disclosure, the term “food raw material” refers to an ingredient or a raw material that is used for producing, for example, foods such as processed foods and health foods as well as supplements. In one or more embodiments, examples of the form of the food raw material include an aqueous solution, a concentrated liquid, and a dried product.

In one or more embodiments, the extract composition of the present disclosure may contain an extract of at least one plant selected from the group consisting of lemon balm, rosemary, fenugreek, and hibiscus.

Method of Decomposing Maillard Reaction Product

In an aspect, the present disclosure relates to a method of decomposing a Maillard reaction product, the method including administering an extract of fennel to a living body. In one or more embodiments, the method of decomposing a Maillard reaction product of the present disclosure may further include administering, to a living body, an extract of at least one plant selected from the group consisting of lemon balm, rosemary, fenugreek, and hibiscus. In an aspect, the present disclosure relates to a method of decomposing a Maillard reaction product, the method including allowing a human or a non-human organism to take in an extract of fennel. In one or more embodiments, the method of decomposing a Maillard reaction product of the present disclosure may use, instead of the above-mentioned extract, a Maillard reaction product-decomposing agent, a beverage composition, a food composition, a functional food, a cosmetic, a supplement, a quasi drug, or a pharmaceutical product of the present disclosure.

The present disclosure may relate to one or more embodiments described below:

(1) A Maillard reaction product-decomposing agent containing an extract of fennel as an active ingredient.
(2) The Maillard reaction product-decomposing agent according to item (1), the agent further containing an extract of at least one of lemon balm and rosemary.
(3) The Maillard reaction product-decomposing agent according to item (1) or (2), wherein the extract is at least one of a water extract and an organic solvent extract.
(4) The Maillard reaction product-decomposing agent according to any one of items (1) to (3), wherein the agent contains, as an active ingredient, a dried product of the extract.
(5) A beverage composition, a food composition, a functional food, a cosmetic, a supplement, a quasi drug, or a pharmaceutical product that contains, as an active ingredient, a Maillard reaction product-decomposing agent according to any one of items (1) to (4).
(6) A beverage composition, a food composition, a functional food, a cosmetic, a supplement, a quasi drug, or a pharmaceutical product for enhancing a Maillard reaction product-decomposing activity, each of which contains an extract of fennel as an active ingredient.
(7) A method of producing a Maillard reaction product-decomposing agent, the method including solvent-extracting fennel.
(8) A method of producing a beverage composition, a food composition, a functional food, a cosmetic, a supplement, a quasi drug, or a pharmaceutical product, the method including adding an extract of fennel to a beverage composition, a food composition, a functional food, a cosmetic, a supplement, a quasi drug, or a pharmaceutical product in order to provide it with a Maillard reaction product-decomposing effect.

Hereinafter, the present disclosure is further described using examples. However, the present disclosure shall not be interpreted to be limited to the following examples.

### Examples

Preparation of Plant Extracts

After 2 g of dry powder of each plant indicated in Table 1 below was extracted with 40 ml of distilled water at 80° C. for one hour, it was cooled to a normal temperature. Thereafter, it was filtrated and the filtrate thus obtained was used as a plant extract.

After 5 mL of resultant filtrate was put into an aluminum tray and then was dried for four hours in an incubator set at 110° C., the remaining amount thereof was measured to calculate the solid content concentration of each sample. The results are indicated in Table 1 below.

AGEs Cross-Link Breaking Effect Confirmatory Test 1

The test was carried out according to the description in Sara Vasan et al. (Nature, 382, pp 275-278 (1996)).

As a substrate, 1-phenyl-1,2-propanedione (PPD), which is a reaction substrate for an AGEs cross-linking model having an α-diketone structure, was used while N-phenacylthiazolium bromide (PTB) was used as a positive control.

A plant extract, 10 mmol/L of PPD, and 0.2 mol/L of phosphate buffer solution (pH 7.4) were mixed together at a ratio of 5:1:4 and then were reacted at 37° C. for eight hours (n=3). After completion of the reaction, hydrochloric acid was added thereto to stop the reaction.

The reaction solution was centrifuged at 20° C. and 3,000×g for ten minutes and then the amount of benzoic acid contained in the supernatant was analyzed by reverse-phase HPLC. The amount of benzoic acid contained in the reaction solution was determined by subtracting the amount of benzoic acid contained in the sample, which was measured separately. Since 1 mol of PPD produces 1 mol of benzoic acid, the cross-link breaking rate was calculated by the following formula. The results are indicated in Table 1 below.

Cross-link breaking rate (%)={(A−B)/C}×100

A: The amount of benzoic acid contained in the reaction solution
B: The amount of benzoic acid contained in the sample
C: The amount of PPD provided for the reaction (the amount of the substrate)

As a control, a test was carried out using 10 mmol/L of PTB instead of the plant extract.

TABLE 1

| | Solid Content | Cross-Link Breaking Rate (%) |
|---|---|---|
| Common Name | Concentration | (Mean Value ± |
| (Part to be Used) | (mg/mL) | Standard Deviation) |
| Fennel (Seeds) | 1.15 | 39.00 ± 4.30 |
| Lemon Balm (Leaves) | 1.32 | 29.60 ± 2.14 |
| Rosemary (Leaves, Stems) | 1.11 | 26.58 ± 0.25 |
| PTB | — | 20.47 ± 0.29 |

As indicated in Table 1, fennel, lemon balm, and rosemary each exhibited a higher cross-link breaking rate than that of PTB that has been reported as a compound that breaks cross-linked structures associated with AGEs Particularly, fennel exhibited a cross-link breaking rate nearly twice as high as that of PTB.

AGEs Cross-Link Breaking Effect Confirmatory Test 2

The test was carried out according to the description in Sara Vasan et al. (Nature, 382, pp 275-278 (1996)).

300 μL/well of SuperBlock T20 (PBS) Blocking Buffer was added to a commercially available 96-well microplate precoated with collagen, which then was reacted at 37° C. for one hour. After the reaction, it was washed with 0.05% polyethylene (20) sorbitan monolaurate (Tween 20)/PBS(−), and then AGEs modified bovine serum albumin (AGE-BSA) was added to each well of the microplate, which then was subjected to a crosslinking reaction at 37° C. for four hours. After the reaction, it was washed with Tween 20/PBS(−). Thereafter, a plant extract was added to each well and thereby it was subjected to a cross-link breaking (cleaving) reaction at 37° C. for 18 hours. After that, it was washed with Tween 20/PBS(−) and a primary antibody (anti-albumin bovine serum rabbit-polyclonal) was added thereto, which then was reacted at room temperature for 30 minutes. Further, after it was washed with Tween 20/PBS(−), a secondary antibody (goat anti-rabbit IgG horseradish peroxidase (IMP) conjugate) was added thereto, which then was reacted at room temperature for 30 minutes. Thereafter, it was washed with Tween 20/PBS(−), and TMB One Component HRP Microwell Substrate was added thereto. Furthermore, a PEG6000 solution was added thereto, which then was reacted for 20 minutes. As a reaction stop solution, 1N HCl was added thereto, and the absorbance at 450 nm (dominant wavelength)/630 nm (complementary wavelength) was measured with a microplate reader. The amount of AGE-BSA that remained was determined from a calibration curve in which the amount of AGE-BSA was varied. Furthermore, the AGE-BSA decomposition rate (Collagen cross-link decomposition rate) was determined using the following formula. The results are indicated in Table 2 below.

AGE-BSA decomposition rate={(Amount of AGE-BSA added−Amount of remaining AGE-BSA)/Amount of AGE-BSA added}×100

As a positive control, the same test was carried out using, instead of the plant extract, punicalagin that has been known to have a Maillard reaction activity-inhibiting effect.

TABLE 2

| | Amount of Remaining | Collagen Cross-Link |
|---|---|---|
| Common Name | AGE-BSA | Decomposition Rate |
| (Part to be Used) | (ng) | (%) |
| Fennel (Seeds) | 812.4 | 18.8 |
| Lemon Balm (Leaves) | 373 | 62.7 |
| Fenugreek (Seeds) | 818.8 | 18.1 |
| Punicalagin | 343.3 | 65.7 |

As shown in Table 2, it was confirmed that fennel, lemon balm, and fenugreek each have a collagen cross-link breaking activity. It was confirmed that in particular, lemon balm has a collagen cross-link breaking activity equivalent to that of punicalagin.

1. A Maillard reaction product-decomposing agent, comprising an extract of fennel as an active ingredient.

2. The Maillard reaction product-decomposing agent according to claim 1, further comprising an extract of at least one of lemon balm and rosemary.

3. The Maillard reaction product-decomposing agent according to claim 1, wherein the extract is at least one of a water extract and an organic solvent extract.

4. The Maillard reaction product-decomposing agent according to claim 1, wherein the agent comprises, as an active ingredient, a dried product of the extract.

5. A beverage composition, a food composition, a functional food, a cosmetic, a supplement, a quasi drug, or a pharmaceutical product, comprising, as an active ingredient, a Maillard reaction product-decomposing agent according to claim 1.

6. The beverage composition, a food composition, a functional food, a cosmetic, a supplement, a quasi drug, or a pharmaceutical product according to claim 5, further comprising an extract of at least one of lemon balm and rosemary.

7. The beverage composition, a food composition, a functional food, a cosmetic, a supplement, a quasi drug, or a pharmaceutical product according to claim 6, wherein the extract is at least one of a water extract and an organic solvent extract.

8. A method of producing a Maillard reaction product-decomposing agent, comprising solvent-extracting fennel.

9. The method according to claim 8, further comprising solvent-extracting at least one plant selected from the group consisting of lemon balm and rosemary.

10. The method according to claim 9, further comprising drying the extract obtained by the solvent-extracting

11. A method of producing a beverage composition, a food composition, a functional food, a cosmetic, a supplement, a quasi drug, or a pharmaceutical product according to claim 5, the method comprising adding an extract of fennel to a beverage composition, a food composition, a functional food, a cosmetic, a supplement, a quasi drug, or a pharmaceutical product in order to provide it with a Maillard reaction product-decomposing effect.

12. The method according to claim 11, further comprising adding an extract of at least one of lemon balm and rosemary to the beverage composition, the food composition, the functional food, the cosmetic, the supplement, the quasi drug, or the pharmaceutical product.

13. A method of decomposing a Maillard reaction product, the method comprising administering, to a living body, a Maillard reaction product-decomposing agent,
wherein the agent comprises an extract of fennel as an active ingredient.

14. The method according to claim 13, wherein the agent further comprises an extract of at least one of lemon balm and rosemary.

15. The method according to claim 14, wherein the agent further comprises an extract of fenugreek.

16. The method according to claim 13, wherein the extract is at least one of a water extract and an organic solvent extract.

17. The method according to claim 13, wherein the extract is a dried product.
